(19) United States
(12) Patent Application Publication
(10) Pub. No.: US 2024/0382111 A1
(43) Pub. Date: Nov. 21, 2024

(54) METHOD OF DETECTING GAIT PATTERN BASED ON DEEP-LEARNING AND COMPUTER PROGRAM PERFORMING THE SAME

(71) Applicant: REMO INC., Seoul (KR)

(72) Inventors: Jung Yoon KIM, Seongnam-si (KR); Hyeon Seok YOU, Seoul (KR); Eun Kyung BAE, Seoul (KR)

(21) Appl. No.: 18/773,762

(22) Filed: Jul. 16, 2024

Related U.S. Application Data
(63) Continuation of application No. PCT/KR2022/014152, filed on Sep. 22, 2022.

(30) Foreign Application Priority Data
Feb. 24, 2022 (KR) 10-2022-0024049

(51) Int. Cl. A61B 5/11; A61B 5/00

(52) U.S. Cl. CPC A61B 5/112; A61B 5/1128; A61B 5/7221; A61B 5/7246; A61B 5/7267; A61B 5/7271

(57) ABSTRACT

A computer program stored on a computer-readable storage medium may be provided.

The computer program stored on a computer-readable storage medium, wherein the computer program may include instructions executable by a processor to: obtain 2D image data of a detection target and extract 3D coordinates based on the 2D image data, wherein the 3D coordinates comprise key points for left and right hip joints, left and right ankles, and pelvis of the detection target; determine the number of steps per minute (cadence) of the detection target; extract a first target angle pattern, which is an angle pattern of the left or right hip joint key point; determine a first reference angle pattern, which is an angle pattern corresponding to the number of steps per minute of the detection target from among a plurality of previously collected angle patterns; generate first filtering data by performing pattern matching (cross-correlation) on the first target angle pattern and the first reference angle pattern; extract a target distance pattern, which is a distance pattern in a gait direction between the left and right ankle key points; determine a reference distance pattern, which is a distance pattern corresponding to the number of steps per minute of the detection target from among a plurality of previously collected distance patterns; generate second filtering data by performing pattern matching on the target distance pattern and the reference distance pattern; determine a gait cycle satisfying both the first and second filtering data; determine whether a difference between maximum and minimum values of a vertical distance between the pelvis key point and the left or right ankle key point in a target distance pattern graph within the gait cycle is greater than or equal to a reference value; determine that a gait within the gait cycle is a valid gait when the difference between the maximum and minimum values of the vertical distance is greater than or equal to the reference value; and determine heel-strike and toe-off points based on a position and slope on the target distance pattern graph within the gait cycle when the gait within the gait cycle is determined to be a valid gait.

### CROSS-REFERENCE TO RELATED APPLICATIONS

This application is a Continuation of Application No. PCT/KR2022/014152, filed on Sep. 22, 2022, which in turn claims the benefit of Korean Patent Application No. 10-2022-0024049, filed on Feb. 24, 2022. The entire disclosures of all these applications are hereby incorporated by reference.

### TECHNICAL FIELD

The present invention relates to a method of detecting a gait pattern and a computer program for performing the same, and more particularly, to a method of extracting three-dimensional coordinates based on two-dimensional image data and detecting a gait pattern (gait cycle and gait event) based on the extracted three-dimensional coordinates, and a computer program for performing the same.

### BACKGROUND ART

Recently, due to the aging population, the number of elderly people complaining of gait abnormalities caused by nerve and muscle damage is increasing. Accordingly, there is a need for a method of collecting bio-signal data that can easily diagnose and predict musculoskeletal diseases.

Referring to FIG. 1, gait motion is the most basic movement for musculoskeletal diagnosis and has a general pattern. That is, a gait cycle is divided into a stance phase and a swing phase, and consists of gait events such as heel strike (or initial contact) and toe off.

In the past, a technology was designed to detect a gait cycle and foot pressure of a detection target by using equipment that measures a ground reaction force to detect gait cycle and gait events. However, in this case, expensive equipment is required to measure a physical ground reaction force, and there is a spatial limitation that the measurement needs to be made in a space equipped with such equipment.

### DESCRIPTION OF EMBODIMENTS

### Technical Problem

Provided are a method and program for detecting a gait pattern of a detection target based on analysis of three-dimensional coordinates extracted from two-dimensional image data without equipment measuring a ground reaction force.

### Solution to Problem

As an embodiment of the present disclosure, a computer program stored on a computer-readable storage medium may be provided.

The computer program stored on a computer-readable storage medium, wherein the computer program may include instructions executable by a processor to: obtain 2D image data of a detection target and extract 3D coordinates based on the 2D image data, wherein the 3D coordinates comprise key points for left and right hip joints, left and right ankles, and pelvis of the detection target; determine the number of steps per minute (cadence) of the detection target; extract a first target angle pattern, which is an angle pattern of the left or right hip joint key point; determine a first reference angle pattern, which is an angle pattern corresponding to the number of steps per minute of the detection target from among a plurality of previously collected angle patterns; generate first filtering data by performing pattern matching (cross-correlation) on the first target angle pattern and the first reference angle pattern; extract a target distance pattern, which is a distance pattern in a gait direction between the left and right ankle key points; determine a reference distance pattern, which is a distance pattern corresponding to the number of steps per minute of the detection target from among a plurality of previously collected distance patterns; generate second filtering data by performing pattern matching on the target distance pattern and the reference distance pattern; determine a gait cycle satisfying both the first and second filtering data; determine whether a difference between maximum and minimum values of a vertical distance between the pelvis key point and the left or right ankle key point in a target distance pattern graph within the gait cycle is greater than or equal to a reference value; determine that a gait within the gait cycle is a valid gait when the difference between the maximum and minimum values of the vertical distance is greater than or equal to the reference value; and determine heel-strike and toe-off points based on a position and slope on the target distance pattern graph within the gait cycle when the gait within the gait cycle is determined to be a valid gait.

The target distance pattern graph within the gait cycle according to an embodiment of the present disclosure may comprise two positive (+) peaks and one negative (−) peak, the target distance pattern graph may comprise (a) a section between a first peak, which is a negative peak closest to a point in time before the gait cycle, and a second peak, which is a first positive peak within the gait cycle, (b) a section between the second peak and a third peak, which is a negative peak within the gait cycle, and (c) a section between the third peak and a fourth peak, which is a last positive peak within the gait cycle, and the determine whether a difference between maximum and minimum values of a vertical distance may comprise determining whether the difference between the maximum and minimum values of the vertical distance in each of sections (a) to (c) is greater than or equal to a reference value.

The reference value according to an embodiment of the present disclosure May 30 mm (millimeters).

The target distance pattern graph within the gait cycle according to an embodiment of the present disclosure may comprise two positive (+) peaks and one negative (−) peak, the target distance pattern graph may comprise (a) a section between a first peak, which is a negative peak closest to a point in time before the gait cycle, and a second peak, which is a first positive peak within the gait cycle, (b) a section between the second peak and a third peak, which is a negative peak within the gait cycle, and (c) a section between the third peak and a fourth peak, which is a last positive peak within the gait cycle.

The determine heel-strike and toe-off points according to an embodiment of the present disclosure may comprise: determining a point at which a forward slope of the target distance pattern graph in section (c) is 95% or a point at which a slope of the forward slope in a section between 70% and 95% is 20% of a maximum forward slope as a heel strike; and determining a point at which a reverse slope of the target distance pattern graph in section (c) is 85% or a point at which a slope of the reverse slope in a section between 70% and 85% is 20% of a maximum reverse slope as a toe off.

The computer program according to an embodiment of the present disclosure may further comprise instructions executable by a processor to: determine a point at which a forward slope of the target distance pattern graph in section (a) is 95% or a point at which a slope of the forward slope in a section between 70% and 95% is 20% of a maximum forward slope as a heel strike; and determine a section between the heel strike in section (a) and the heel strike in section (c) as a final gait cycle.

The computer program may further comprise instructions executable by a processor to: extract a second target angle pattern, which is an angle pattern of a right or left hip joint key point, wherein the right or left hip joint key point is a hip joint key point on an opposite side of a hip joint that is a target of the first target angle pattern; determine a second reference angle pattern for performing pattern matching with the second target angle pattern from among the plurality of angle patterns; and generate third filtering data by performing pattern matching on the second target angle pattern and the second reference angle pattern, wherein the determine a gait cycle comprises determining a gait cycle that satisfies all of the first to third filtering data.

### BRIEF DESCRIPTION OF DRAWINGS

FIG. 1 is a view for explaining a general gait cycle and a gait event.

FIGS. 2 and 3 are views for explaining a detection device according to some embodiments of the present invention.

FIGS. 4 and 5 are views for explaining a configuration of a processor of a detection device.

FIG. 6 is a view for explaining a method of detecting a gait cycle and a gait event according to some embodiments of the present invention.

FIG. 7 is a view for explaining a method of determining a gait cycle according to some embodiments of the present invention.

FIG. 8 is a view for explaining an angular pattern of a hip joint key point according to some embodiments of the present invention.

FIG. 9A is an exemplary view of a target angle pattern graph of a detection target, FIG. 9B is an exemplary view of a reference angle pattern graph according to the number of steps per minute, and FIG. 9C is an exemplary view of a pattern matching result of a target angle pattern and a reference angle pattern.

FIG. 10 is a view for explaining a distance pattern between left and right ankle key points according to some embodiments of the present invention.

FIG. 11A is an exemplary view of a target distance pattern graph of a detection target, FIG. 11B is an exemplary view of a reference distance pattern graph according to the number of steps per minute, and FIG. 11C is an exemplary view of a pattern matching result of a target distance pattern and a reference distance pattern.

FIG. 12 is an exemplary view of a gait cycle and sections (a) to (c) determined according to some embodiments of the present invention.

FIG. 13 is a view for explaining a method of determining the validity of a gait cycle according to some embodiments of the present invention.

FIGS. 14A to 14C are views for explaining a vertical distance between a pelvic key point and an ankle key point.

FIG. 15 is a view for explaining a method of determining the validity of a gait based on the vertical distance of FIG. 14A.

FIG. 16 is a view for explaining a method of determining a gait event according to some embodiments of the present invention.

FIG. 17 is a view for explaining a method of determining a heel-strike point in a distance graph between left and right ankle key points.

FIG. 18 is a view for explaining a method of determining a toe-off point in a distance graph between left and right ankle key points.

### MODE OF DISCLOSURE

Hereinafter, embodiments of the present invention will be described with reference to the drawings. In the following description, descriptions of a well-known technical configuration in relation to a lead implantation system for a deep brain stimulator will be omitted. For example, descriptions of the configuration/structure/method of a device or system commonly used in deep brain stimulation, such as the structure of an implantable pulse generator, a connection structure/method of the implantable pulse generator and a lead, and a process for transmitting and receiving electrical signals measured through the lead with an external device, will be omitted. Even if these descriptions are omitted, one of ordinary skill in the art will be able to easily understand the characteristic configuration of the present invention through the following description.

Recently, due to the aging population, the number of elderly people complaining of gait abnormalities caused by nerve and muscle damage is increasing. Accordingly, there is a need for a method of collecting bio-signal data that can easily diagnose and predict musculoskeletal diseases.

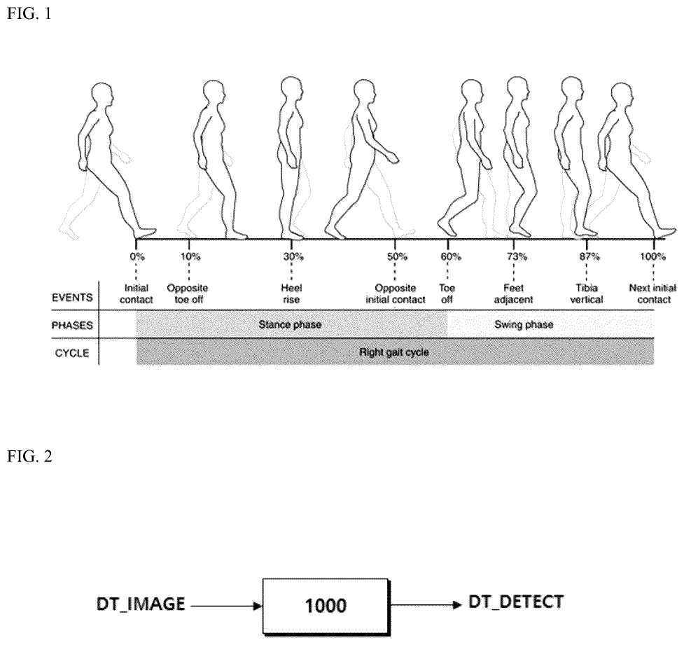

Referring to FIG. 1, gait motion is the most basic movement for musculoskeletal diagnosis and has a general pattern. That is, a gait cycle is divided into a stance phase and a swing phase, and consists of gait events such as heel strike (or initial contact) and toe off.

In the past, a technology was designed to detect a gait cycle and foot pressure of a detection target by using equipment that measures a ground reaction force to detect gait cycle and gait events. However, in this case, expensive equipment is required to measure a physical ground reaction force, and there is a spatial limitation that the measurement needs to be made in a space equipped with such equipment.

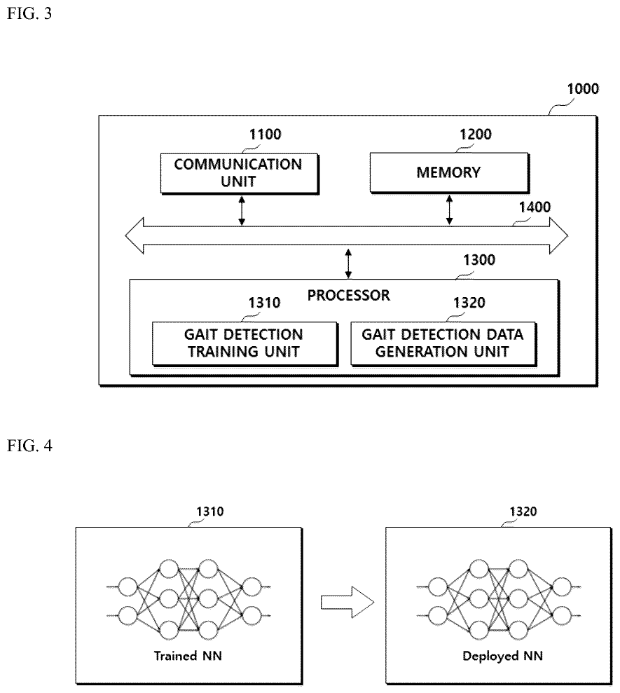

FIGS. 2 and 3 are views for explaining a detection device according to some embodiments of the present invention.

Referring to FIG. 2, a detection device 1000 may obtain two-dimensional image data DT_IMAGE including a gait image of a detection target, and output detection data DT_DETECT including a gait cycle and/or gait event of the detection target based on the two-dimensional image data DT_IMAGE. For example, the two-dimensional image data DT_IMAGE may be data received from a user terminal such as a smartphone or a tablet PC. As another example, the two-dimensional image data DT_IMAGE may be obtained from a separate hardware device equipped with a camera. As another example, the two-dimensional image data DT_IMAGE may be data directly obtained from a camera module (not shown) mounted on the detection device 1000.

The detection device 1000 according to an embodiment corresponds to a computing device having various processing functions such as functions for generating a neural network, training (or learning) a neural network, or retraining a neural network. For example, the detection device 1000 may be implemented by various types of devices such as a personal computer (PC), a server device, and a mobile device.

Referring to FIG. 3, the detection device 1000 according to some embodiments of the present invention may include a communication unit 1100, a memory 1200, and a processor 1300.

The communication unit 1100 may connect the detection device 1000 to a user terminal or an external device for communication. The communication unit 1100 may support a certain short-range communication protocol (e.g., wireless fidelity (Wi-Fi), bluetooth (BT), or near field communication (NFC)), certain network communication (e.g., Internet, local area network (LAN), wire area network (WAN), telecommunication network, cellular network, satellite network, plain old telephone service (POTS), etc.), or a wired communication protocol (e.g., universal serial bus (USB), high definition multimedia interface (HDMI), etc.).

The memory 1200 may store commands or data received from or generated by other components of the detection device 1000. According to some embodiments, the memory 1200 may be composed of a program memory and data memories, and the program memory may store a program for controlling a general operation of the detection device 1000. According to some embodiments, the memory 1200 may further include an external memory such as a compact flash (CF), secure digital (SD), micro-SD, mini-SD, extreme digital (xD), and a memory stick. In addition, the memory 1200 may include a disk storage device such as a hard disk drive (HDD) and a solid state disk (SSD).

The processor 1300 may perform operations to extract three-dimensional coordinates based on the two-dimensional image data DT_IMAGE of the detection target through a gait detection training unit 1310 and a gait detection data generation unit 1320, and to detect a gait cycle and a gait event of the detection target based on the three-dimensional coordinates.

According to some embodiments, the processor 1300 may include an artificial intelligence trained model that is trained and operated using an artificial neural network. The artificial neural network is a computational system that imitates the way the human brain processes information.

A deep neural network is one method of implementing an artificial neural network and may include multiple layers. For example, the deep neural network includes an input layer to which input data is applied, an output layer that outputs a result value derived through prediction based on the input data based on learning, and multiple hidden layers between the input layer and the output layer.

Deep neural networks are classified into convolutional neural networks and recurrent neural networks, depending on the algorithm used to process information.

A method of training an artificial neural network is called deep learning, and as described above, various algorithms such as convolutional neural network and recurrent neural network methods may be used in deep learning.

At this time, training an artificial neural network may refer to determining and updating weights and biases between layers or weights and biases between multiple neurons belonging to different layers from among adjacent layers.

For example, a plurality of hierarchical structures and weights and biases between multiple layers or neurons may be collectively referred to as connectivity of an artificial neural network. Therefore, training an artificial neural network may refer to building and training connectivity.

A data bus 1400 may be a circuit that connects components of the detection device 1000 described above to each other and transmits communication (e.g., control messages) between the components described above.

According to some embodiments, each component of the processor 1300, that is, the gait detection training unit 1310 and gait detection data generation unit 1320, may be a processed neural network. In addition, these are shown separately in the drawing to indicate that they can be functionally and logically separated, and do not necessarily mean that they are physically separate components or implemented in separate code.

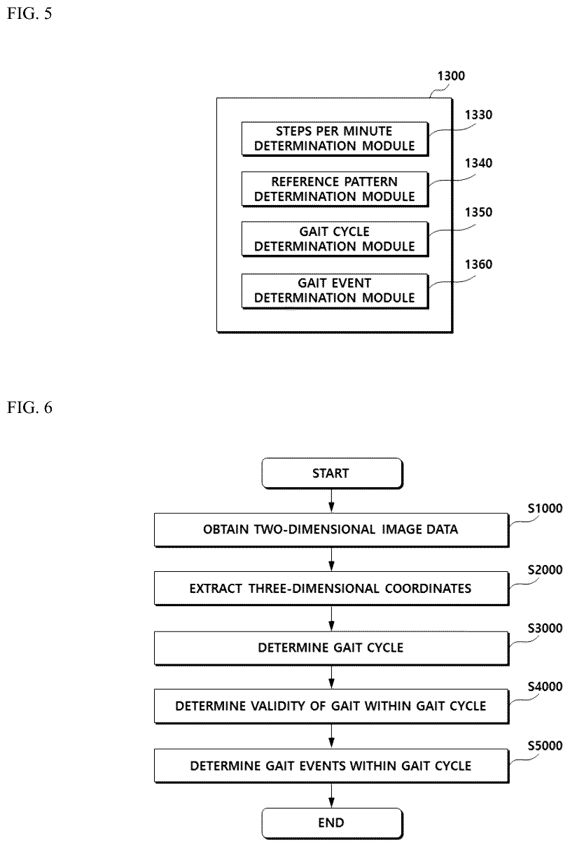

FIGS. 4 and 5 are views for explaining a configuration of a processor of a detection device.

Referring to FIG. 4, the gait detection training unit 1310 of the processor 1300 may repeatedly train (learn) a given initial neural network to generate a trained neural network. Generating a trained neural network may mean determining neural network parameters. Here, the parameters may include various types of data input/output to/from a neural network, such as input/output activations, weights, and biases of the neural network. As the repetitive training of the neural network progresses, the parameters of the neural network may be tuned to compute a more accurate output for a given input.

The gait detection training unit 1310 may transfer the trained neural network to the gait detection data generation unit 1320. The gait detection data generation unit 1320 may be included in a mobile device, an embedded device, etc. The gait detection data generation unit 1320 may be dedicated hardware for driving the neural network.

The gait detection data generation unit 1320 may drive the trained neural network, or may drive a neural network in which the trained neural network is processed (e.g., quantized). The gait detection data generation unit 1320 that drives the processed neural network may be implemented in an independent device separate from the gait detection training unit 1310. However, the present invention is not limited thereto, and the gait detection data generation unit 1320 may also be implemented in the same device as that of the gait detection training unit 1310.

Referring to FIG. 5, the processor 1300 may include a steps per minute determination module 1330, a reference pattern determination module 1340, a gait cycle determination module 1350, and a gait event determination module 1360. According to an embodiment, each of the steps per minute determination module 1330, the reference pattern determination module 1340, the gait cycle determination module 1350, and the gait event determination module 1360 may include the gait detection training unit 1310 and the gait detection data generation unit 1320. According to another embodiment, the steps per minute determination module 1330, the reference pattern determination module 1340, the gait cycle determination module 1350, and the gait event determination module 1360 are shown separately in the drawing to indicate that they can be functionally and logically separated, and do not necessarily mean that they are physically separate components or implemented in separate code.

The steps per minute determination module 1330 may determine the number of steps per minute (cadence) of a detection target. According to an embodiment, the steps per minute determination module 1330 may determine the number of steps per minute of the detection target based on two-dimensional image data DT_IMAGE. According to another embodiment, the steps per minute determination module 1330 may determine the number of steps per minute of the detection target based on extracted three-dimensional coordinates.

The reference pattern determination module 1340 may determine a reference pattern based on the number of steps per minute of the detection target determined by the steps per minute determination module 1330. For example, the reference pattern determination module 1340 may determine a pattern corresponding to the number of steps per minute of the detection target from among multiple patterns stored in the memory 1200 as a reference pattern.

The gait cycle determination module 1350 may generate first and second filtering data based on three-dimensional coordinates, and determine a gait cycle that satisfies both the first and second filtering data.

The gait event determination module 1360 may determine a gait event in the gait cycle based on a distance between left and right ankle key points before and after the determined gait cycle.

The specific operations of the steps per minute determination module 1330, the reference pattern determination module 1340, the gait cycle determination module 1350, and the gait event determination module 1360 will be described in detail later with reference to FIG. 6 and below.

FIG. 6 is a view for explaining a method of detecting a gait cycle and a gait event according to some embodiments of the present invention.

In operation S1000, the detection device 1000 may obtain the two-dimensional image data DT_IMAGE of a detection target. The two-dimensional image data DT_IMAGE may include a gait image of the detection target.

In operation S2000, the detection device 1000 may extract three-dimensional coordinates based on the two-dimensional image data. According to an embodiment, the three-dimensional coordinates may include key points for the left and right hip joints, left and right ankles, and pelvis of the detection target. Each key point may be extracted in the form of a point or a line.

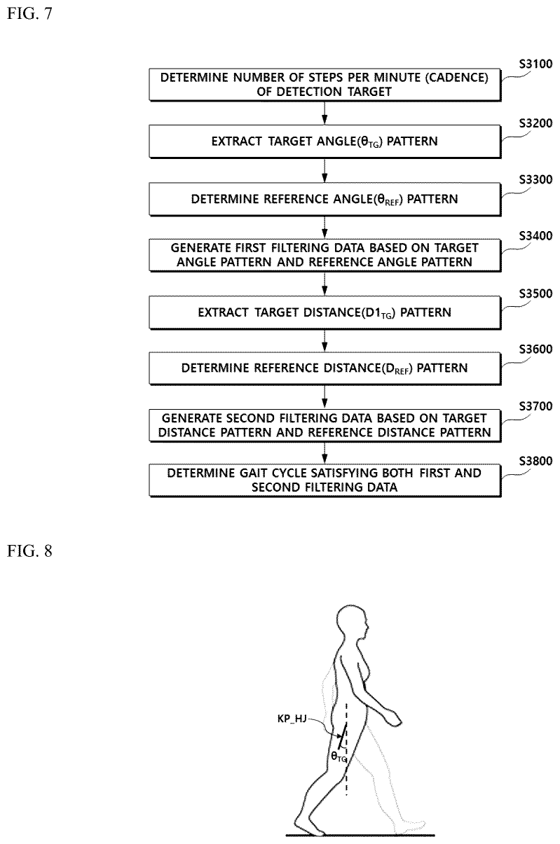

In operation S3000, the detection device 1000 may determine a gait cycle based on the extracted three-dimensional coordinates. The operation of determining the gait cycle will be described in detail later with reference to FIGS. 7 to 12.

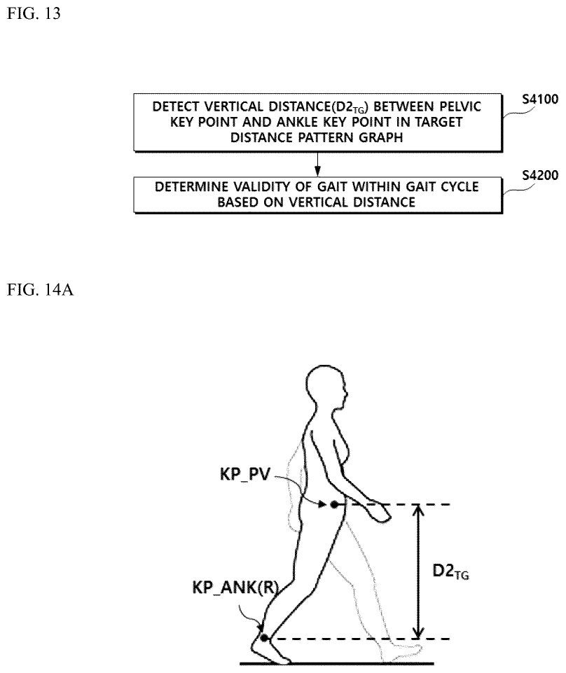

In operation S4000, the detection device 1000 may determine the validity of a gait of the detection target in the determined gait cycle. That is, the detection device 1000 may determine whether the gait within the gait cycle is a valid gait or a gait (or noise) that is not suitable for determining the gait cycle. The operation of determining the validity of a gait will be described in detail later with reference to FIGS. 13 to 15.

In operation S5000, when the gait of the detection target within the gait cycle is determined to be valid, the detection device 1000 may determine gait events of the gait cycle, that is, a heel-strike (HS) point and a toe-off (TO) point. The operation of determining the gait events will be described in detail later with reference to FIGS. 16 to 18.

FIG. 7 is a view for explaining a method of determining a gait cycle according to some embodiments of the present invention. Referring to FIG. 7, the detection device 1000 according to some embodiments of the present invention may determine the gait cycle based on image data of the detection target. Operation S3000 of FIG. 6 may include operations S3100 to S3800.

In operation S3100, the steps per minute determination module 1330 may determine the number of steps per minute of the detection target. According to an embodiment, the steps per minute determination module 1330 may determine the number of steps per minute of the detection target based on three-dimensional coordinates. According to another embodiment, the steps per minute determination module 1330 may determine the number of steps per minute of the detection target based on the two-dimensional image data DT_IMAGE.

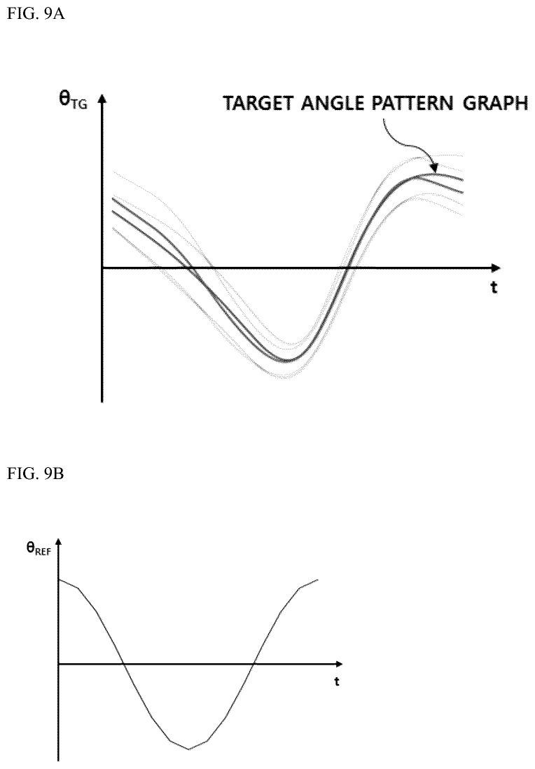

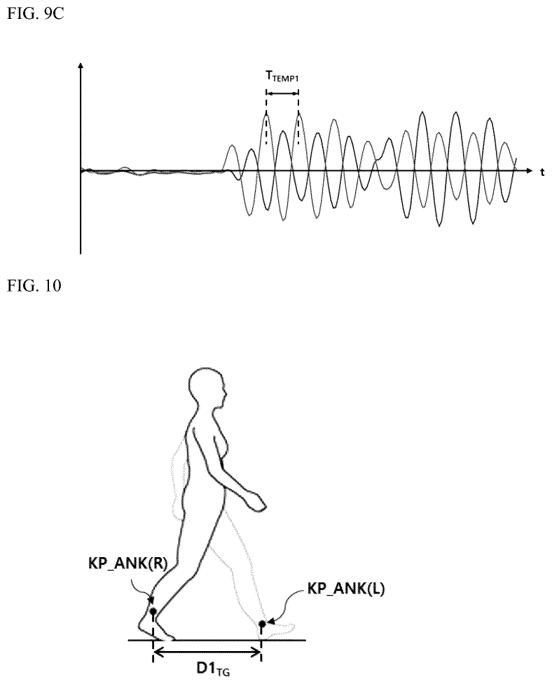

FIG. 8 is a view for explaining an angular pattern of a hip joint key point according to some embodiments of the present invention, FIG. 9A is an exemplary view of a target angle pattern graph of a detection target, FIG. 9B is an exemplary view of a reference angle pattern graph according to the number of steps per minute, and FIG. 9C is an exemplary view of a pattern matching result of a target angle pattern and a reference angle pattern.

Referring to FIGS. 7 to 9C, in operation S3200, the gait cycle determination module 1350 may extract a target angle pattern for a target angle θ_{TG} based on three-dimensional coordinates, in detail, left and/or right hip joint key points KP_HJ. For convenience of explanation, it is assumed that the target angle pattern is extracted based on the right hip joint key point, and hereinafter, “hip joint key point” may mean the right hip joint key point. The target angle θ_{TG} may mean an angle formed by the hip joint key point KP_HJ and a direction perpendicular to the ground on a sagittal plane of the detection target. In this case, the sagittal plane means a plane that divides the body into left and right, and may be expressed as a longitudinal section. As shown, the hip joint key point KP_HJ may be extracted in the form of a line, and an angle that the hip joint key point KP_HJ makes with a reference line (a line perpendicular to the ground) may be defined as the target angle θ_{TG}. FIG. 9A is an example of the target angle pattern graph of the detection target, and it is assumed that an indicated graph from among multiple graphs is the target angle pattern graph of the detection target.

In operation S3300, the reference pattern determination module 1340 may determine a pattern for a reference angle θ_{REF} to perform pattern matching (cross-correlation) with a pattern for the target angle θ_{TG}. FIG. 9B is an example of a reference angle pattern, and an angle pattern corresponding to the number of steps per minute of the detection target from among a plurality of previously stored angle patterns may be determined as a reference angle pattern.

In operation S3400, the gait cycle determination module 1350 may generate first filtering data based on a target angle pattern and the reference angle pattern. According to an embodiment, the gait cycle determination module 1350 may perform pattern matching on a determined target angle pattern and the reference angle pattern to generate the first filtering data. FIG. 9C is an exemplary view of a pattern matching result of the target angle pattern and the reference angle pattern, and the first filtering data may include information about the pattern matching result and a first temporary gait cycle T_{TEMP1}. That is, a section with an excellent pattern matching result according to a predefined reference may be determined as the first temporary gait cycle T_{TEMP1}.

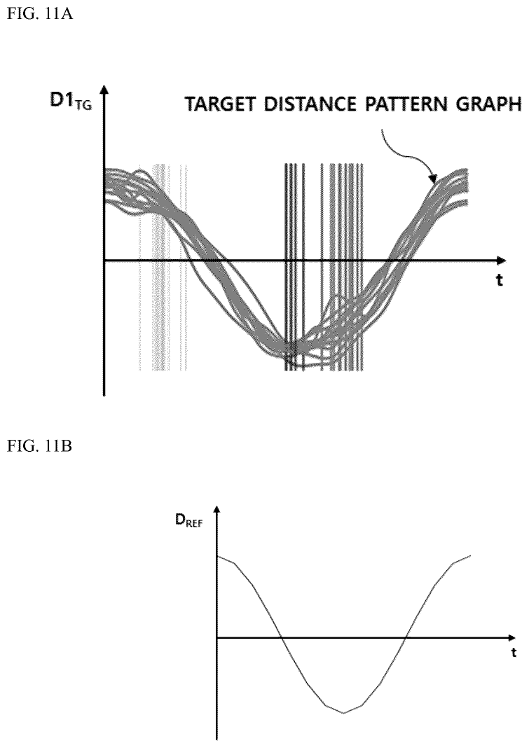

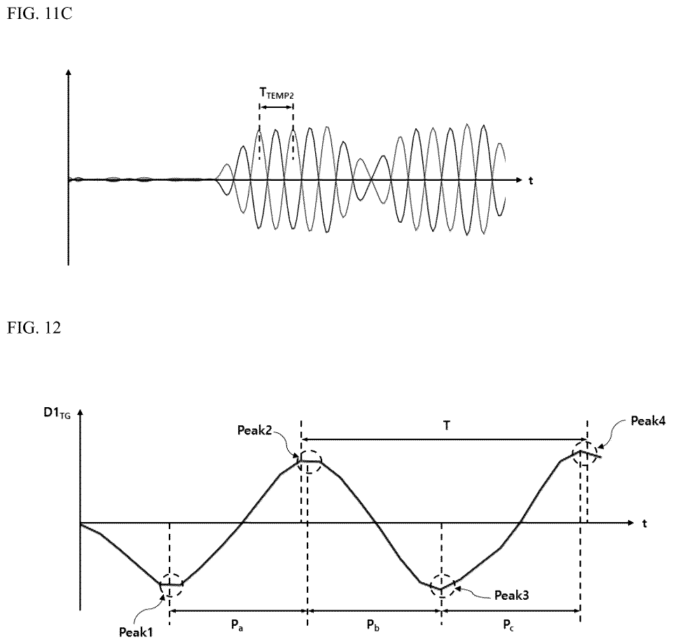

FIG. 10 is a view for explaining a distance pattern between left and right ankle key points according to some embodiments of the present invention, FIG. 11A is an exemplary view of a target distance pattern graph of a detection target, FIG. 11B is an exemplary view of a reference distance pattern graph according to the number of steps per minute, and FIG. 11C is an exemplary view of a pattern matching result of a target distance pattern and a reference distance pattern.

Referring to FIG. 7 and FIGS. 10 to 11C, in operation S3500, the gait cycle determination module 1350 may extract a target angle pattern for a target distance D1_{TG} based on 3D coordinates, in more detail, a left ankle key point KP_ANK(L) and a right ankle key point KP_ANK(R). The target distance D1_{TG} means a distance between the left ankle key point KP_ANK(L) and the right ankle key point KP_ANK(R) of the detection target in the direction of progress. That is, the target distance D1_{TG} means a distance between the left ankle key point KP_ANK(L) and the right ankle key point KP_ANK(R) in the direction of progress of the detection target parallel to the ground. FIG. 11A is an example of the target angle pattern graph of the detection target, and it is assumed that an indicated graph from among multiple graphs is the target angle pattern graph of the detection target. For convenience of explanation, the distance between the left ankle key point KP_ANK(L) and the right ankle key point KP_ANK(R) will be described based on the right ankle key point KP_ANK(R). That is, it will be described that the right ankle key point (KP_ANK(R)) has a positive (+) value when it is located in front in the direction of movement, and a negative (−) value when it is located behind in the direction of movement. However, this is an example, and it is obvious that the present invention can be applied based on the left ankle key point (KP_ANK(L)).

In operation S3600, the reference pattern determination module 1340 may determine a pattern graph for the target distance D1_{TG} and a pattern for a reference distance DREF to perform pattern matching. FIG. 11B is an example of a reference distance DREF, and a distance pattern corresponding to the number of steps per minute of the detection target from among a plurality of previously stored angle patterns may be determined as a reference distance pattern.

In operation S3700, the gait cycle determination module 1350 may generate second filtering data based on a target distance pattern and the reference distance pattern. According to an embodiment, the gait cycle determination module 1350 may perform pattern matching on a determined target distance pattern and the reference distance pattern to generate the second filtering data. FIG. 11C is an exemplary view of a pattern matching result of the target distance pattern and the reference distance pattern, and the second filtering data may include information about the pattern matching result and a second temporary gait cycle T_{TEMP2}. That is, a section with an excellent pattern matching result according to a predefined reference may be determined as the second temporary gait cycle T_{TEMP2}.

FIG. 12 is an exemplary view of a gait cycle and sections (a) to (c) determined according to some embodiments of the present invention.

Referring to FIGS. 7 and 12, in operation S3800, the gait cycle determination module 1350 may determine a gait cycle T. A section satisfying both the first temporary gait cycle T_{TEMP1} included in the first filtering data determined based on the pattern of the target angle θ_{TG} and the second temporary gait cycle T_{TEMP2} included in the second filtering data determined based on the pattern of the target distance D1_{TG} may be determined as the gait cycle T.

As illustrated, the target distance D1_{TG} of the detection target, that is, the determined gait cycle T is expressed in a distance graph between the left ankle key point KP_ANK(L) and the right ankle key point KP_ANK(R). In the graph, the gait cycle T may include two positive (+) peaks (Peak2 and Peak4) and one negative (−) peak (Peak3). Hereafter, for the purpose of explanation, one negative peak before the gait cycle is defined as the first peak Peak1, and three peaks within the gait cycle are defined as the second peak Peak2, the third peak Peak3, and the fourth peak Peak4, respectively. In addition, a section between the first peak Peak1 and the second peak Peak2 is defined as section (a) Pa, a section between the second peak Peak2 and the third peak Peak3 is defined as section (b) Pb, and a section between the third peak Peak3 and the fourth peak Peak4 is defined as section (c) Pc.

Although not shown, according to some embodiments, generating third filtering data by performing pattern matching on an angle pattern of a hip joint key point opposite to the hip joint key point where the first filtering data is to be generated and the reference angle pattern, and determining a gait cycle that satisfies all of the first to third filtering data may be further included. For example, the accuracy of detection may be further improved by determining the first filtering data and the first temporary gait cycle based on the right hip joint key point, determining the third filtering data and the third temporary gait cycle based on the left hip joint key point, and determining the gait cycle that satisfies all of the first to third filtering data.

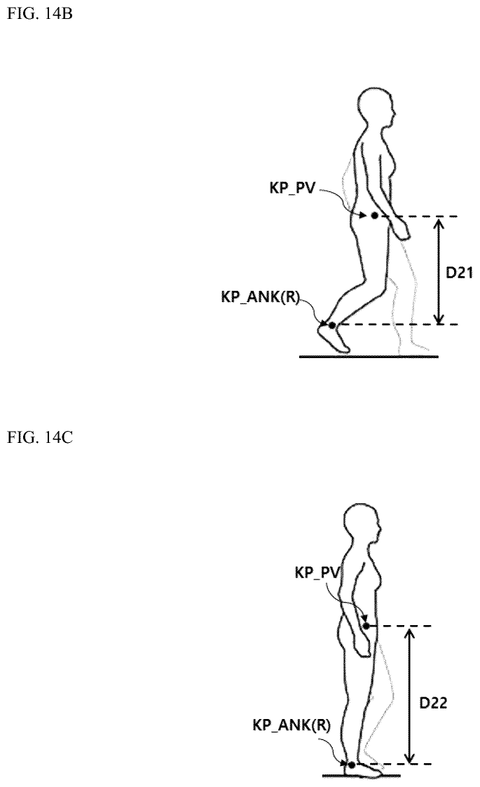

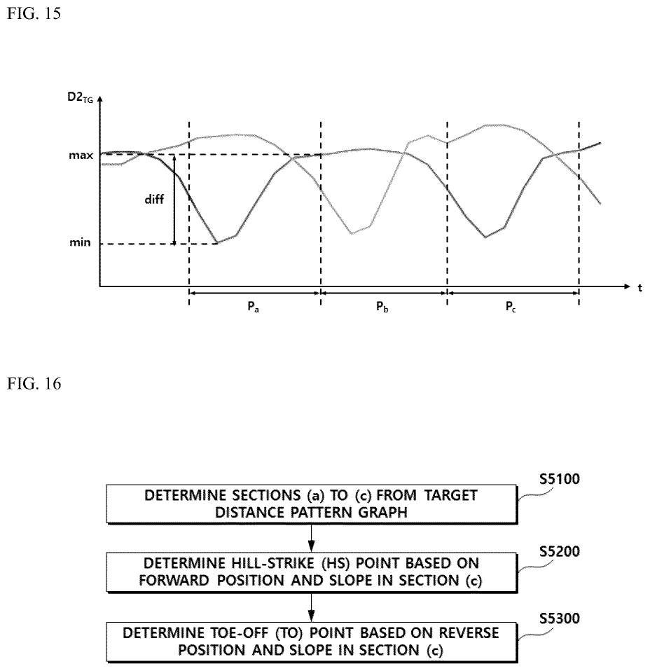

FIG. 13 is a view for explaining a method of determining the validity of a gait cycle according to some embodiments of the present invention, FIGS. 14A to 14C are views for explaining a vertical distance between a pelvic key point and an ankle key point, and FIG. 15 is a view for explaining a method of determining the validity of a gait based on the vertical distance of FIG. 14A.

Referring to FIG. 13, the detection device 1000 according to some embodiments of the present invention may determine the validity of a gait of a detection target within the gait cycle T. Operation S4000 of FIG. 6 may include operations S4100 and S4200.

Referring to FIGS. 13 and 14, in operation S4100, a vertical distance D2_{TG} between a pelvic key point KP_PV and the right ankle key point KP_ANK(R) or the left ankle key point KP_ANK(L) in a target distance pattern graph may be detected. The vertical distance D2_{TG} may mean a height direction of the detection target that is perpendicular to the ground. According to an embodiment, a distance from the ankle key point that has a shorter vertical distance from the pelvic key point KP_PV from among the right ankle key point KP_ANK(R) and the left ankle key point KP_ANK(L) may be defined as the vertical distance D2_{TG}. As another example, a target of the vertical distance D2_{TG} measured in a specific section may be preset as the right ankle key point KP_ANK(R) or the left ankle key point KP_ANK(L).

For example, when a distance between the right ankle key point KP_ANK(R) and the pelvic key point KP_PV of the detection target is set as the vertical distance D2_{TG}, as shown in FIG. 14B, when the right foot is lifted, a vertical distance D21 may be relatively shorter, and when the right foot is in contact with the ground, a vertical distance D22 may be relatively longer.

Referring to FIGS. 13 and 15, in operation S4200, the validity of the gait within the gait cycle T may be determined based on the detected vertical distance D2_{TG}. FIG. 15 is illustrated and described based on a maximum value max and a minimum value min of the vertical distance D2_{TG} in section (a) Pa, but may be substantially applied to section (b) Pb and section (c) Pc.

According to an embodiment, when a difference diff between the maximum value max and minimum value min of the vertical distance D2_{TG} in the gait cycle T or section (a) Pa to section (c) Pc is greater than or equal to a predefined reference value, it can be determined that the gait within the gait cycle T is a valid gait. For example, the reference value may be 30 mm (millimeters).

As another example, when the difference diff between the maximum value max and minimum value min of the vertical distance D2_{TG} is greater than or equal to the reference value in each of section (a) Pa, section (b) Pb, and section (c) Pc, it can be determined that the gait within the gait cycle T is a valid gait. At this time, respective ankle key points that are targets of the vertical distance D2_{TG} in section (a) Pa, section (b) Pb, and section (c) Pc may be set differently. For example, in section (a) Pa, a distance between the right ankle key point KP_ANK(R) and the pelvic key point KP_PV may be measured as the vertical distance D2_{TG}, in section (b) Pb, a distance between the left ankle key point KP_ANK(L) and the pelvic key point KP_PV may be measured as the vertical distance D2_{TG}, and in section (c) Pc, a distance between the right ankle key point KP_ANK(R) and the pelvic key point KP_PV may be measured as the vertical distance D2_{TG} again.

According to some embodiments of the present invention, it can be determined whether the gait cycle T is an invalid gait (e.g., first step, change of direction, etc.) based on the vertical distance D2_{TG} between an ankle key point KP_ANK and the pelvic key point KP_PV. Whether it is a first step can be determined by determining the vertical distance D2_{TG} in the previous section (section (a) Pa) of the gait cycle T, and whether it is a valid step can be accurately determined by checking whether the foot is lifted during a swing phase in section (b) Pb and section (c) Pc included within the gait cycle T.

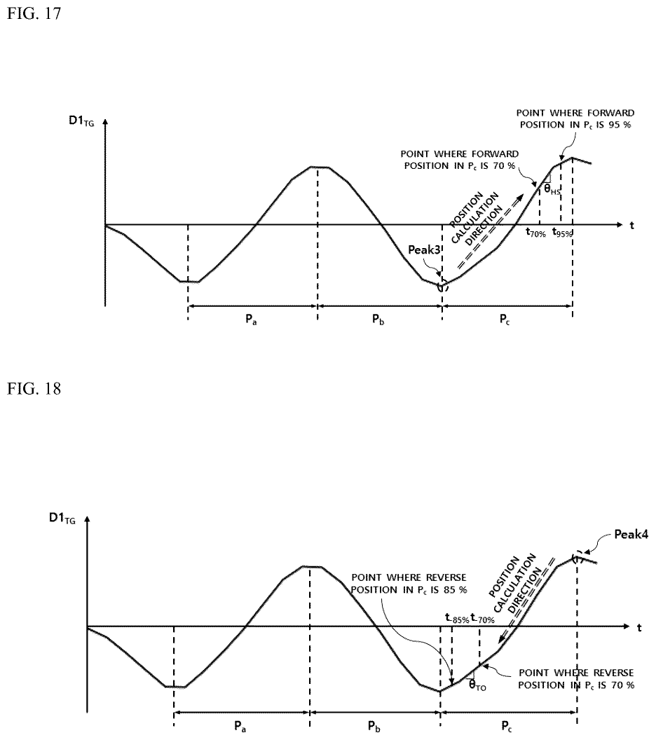

FIG. 16 is a view for explaining a method of determining a gait event according to some embodiments of the present invention, FIG. 17 is a view for explaining a method of determining a heel-strike point in a distance graph between left and right ankle key points, and FIG. 18 is a view for explaining a method of determining a toe-off point in a distance graph between left and right ankle key points.

Referring to FIG. 16, the detection device 1000 according to some embodiments of the present invention may determine a gait event within a gait cycle based on the position and slope of a target distance pattern graph of a detection target. Operation S5000 of FIG. 6 may include operations S5100 to S5300.

In operation S5100, sections (a) to (c) may be determined from the target distance pattern graph of the detection target. The content of determining sections (a) to (c) Pa, Pb, and Pc based on four peaks (Peak1, Peak 2, Peak 3, and Peak 4) is substantially the same as the content described with reference to FIG. 12.

Referring to FIGS. 16 and 17, in operation S5200, a hill-strike (HS) point may be determined based on a forward position and slope in section (c) Pc.

According to an embodiment, a starting point of section (c) Pc, that is, a point where the position on a forward graph of section (c) Pc is 95% from the third peak Peak3, and among points where a forward slope θ_{HS} is 20% or less of a maximum forward slope θ_{HS} in section (c) Pc in a section where the position on the forward graph of section (c) Pc is 70% to 95%, the faster point may be determined as the hill-strike (HS) point. That is, the heel-strike (HS) point, which is an end point of the gait cycle T, may be determined using a position and slope of a pattern graph for the target distance D1_{TG} in section (c) Pc.

According to an embodiment, a starting point of section (a) Pa, that is, a point where the position on a forward graph of section (a) Pa is 95% from the first peak Peak1, and among points where a forward slope θ_{HS} is 20% or less of a maximum forward slope θ_{HS} in section (a) Pa in a section where the position on the forward graph of section (a) Pa is 70% to 95%, the faster point may be determined as the hill-strike (HS) point. That is, the heel-strike (HS) point, which is a starting point of the gait cycle T, may be determined using a position and slope of a pattern graph for the target distance D1_{TG} in section (a) Pa.

According to some embodiments, determining a final gait cycle by using the heel-strike (HS) point determined in section (a) Pa and the heel-strike (HS) point determined in section (c) Pc as the starting point and the end point, respectively, may be further included. That is, an accurate gait cycle may be corrected through gait event detection.

Referring to FIGS. 16 and 18, in operation S5300, the toe-off (TO) point may be determined based on a reverse position and slope in section (c) Pc.

According to an embodiment, an end point of section (c) Pc, that is, a point where the position on a reverse graph of section (c) Pc is 85% from the fourth peak Peak4, and among points where a reverse slope Oro is 20% or less of a maximum reverse slope θ_{TO} in section (c) Pc in a section where the position on the reverse graph of section (c) Pc is 70% to 85%, the faster point (latest point in terms of time) may be determined as the toe-off (TO) point. That is, the position and slope of the pattern graph for the target distance D1_{TG} in section (c) Pc may be used to more accurately determine a gait event, specifically a toe-off point.

The embodiments described above may be implemented by hardware components, software components, and/or any combination thereof. For example, the devices, the methods, and components described in the embodiments may be implemented by using general-purpose computers or special-purpose computers, such as a processor, a controller, an arithmetic logic unit (ALU), a digital signal processor, a microcomputer, a field programmable gate array (FPGA), a programmable logic unit (PLU), a microprocessor, or any other devices which may execute and respond to instructions. A processing apparatus may execute an operating system (OS) and a software application executed in the OS. Also, the processing apparatus may access, store, operate, process, and generate data in response to the execution of software. For convenience of understanding, it may be described that one processing apparatus is used. However, one of ordinary skill in the art will understand that the processing apparatus may include a plurality of processing elements and/or various types of processing elements. For example, the processing apparatus may include a plurality of processors or a processor and a controller. Also, other processing configurations, such as a parallel processor, are also possible.

The software may include computer programs, code, instructions, or any combination thereof, and may construct the processing apparatus for desired operations or may independently or collectively command the processing apparatus. In order to be interpreted by the processing apparatus or to provide commands or data to the processing apparatus, the software and/or data may be permanently or temporarily embodied in any types of machines, components, physical devices, virtual equipment, computer storage mediums, or transmitted signal waves. The software may be distributed over network coupled computer systems so that it may be stored and executed in a distributed fashion. The software and/or data may be recorded in a computer-readable recording medium.

A method according to an embodiment may be implemented as program instructions that can be executed by various computer devices, and recorded on a computer-readable recording medium. The computer-readable recording medium may include program instructions, data files, data structures or a combination thereof. Program instructions recorded on the medium may be particularly designed and structured for embodiments or available to one of ordinary skill in a field of computer software. Examples of the computer-readable recording medium include magnetic media, such as a hard disc, a floppy disc, and magnetic tape; optical media, such as a compact disc-read only memory (CD-ROM) and a digital versatile disc (DVD); magneto-optical media, such as floptical discs; and hardware devices specially configured to store and execute program instructions, such as ROM, random-access memory (RAM), a flash memory, etc. Program instructions may include, for example, high-level language code that can be executed by a computer using an interpreter, as well as machine language code made by a complier.

In concluding the detailed description, those skilled in the art will appreciate that many variations and modifications may be made to the preferred embodiments without substantially departing from the principles of the present invention. Therefore, the disclosed preferred embodiments of the invention are used in a generic and descriptive sense only and not for purposes of limitation.

1. A computer program stored on a computer-readable storage medium, wherein the computer program comprises instructions executable by a processor to:
obtain 2D image data of a detection target and extract 3D coordinates based on the 2D image data, wherein the 3D coordinates comprise key points for left and right hip joints, left and right ankles, and pelvis of the detection target;
determine the number of steps per minute (cadence) of the detection target;
extract a first target angle pattern, which is an angle pattern of the left or right hip joint key point;
determine a first reference angle pattern, which is an angle pattern corresponding to the number of steps per minute of the detection target from among a plurality of previously collected angle patterns;
generate first filtering data by performing pattern matching (cross-correlation) on the first target angle pattern and the first reference angle pattern;
extract a target distance pattern, which is a distance pattern in a gait direction between the left and right ankle key points;
determine a reference distance pattern, which is a distance pattern corresponding to the number of steps per minute of the detection target from among a plurality of previously collected distance patterns;
generate second filtering data by performing pattern matching on the target distance pattern and the reference distance pattern;
determine a gait cycle satisfying both the first and second filtering data;
determine whether a difference between maximum and minimum values of a vertical distance between the pelvis key point and the left or right ankle key point in a target distance pattern graph within the gait cycle is greater than or equal to a reference value;
determine that a gait within the gait cycle is a valid gait when the difference between the maximum and minimum values of the vertical distance is greater than or equal to the reference value; and
determine heel-strike and toe-off points based on a position and slope on the target distance pattern graph within the gait cycle when the gait within the gait cycle is determined to be a valid gait.

2. The computer program stored on a computer-readable storage medium of claim 1, wherein the target distance pattern graph within the gait cycle comprises two positive (+) peaks and one negative (−) peak,
the target distance pattern graph comprises (a) a section between a first peak, which is a negative peak closest to a point in time before the gait cycle, and a second peak, which is a first positive peak within the gait cycle, (b) a section between the second peak and a third peak, which is a negative peak within the gait cycle, and (c) a section between the third peak and a fourth peak, which is a last positive peak within the gait cycle, and
the determine whether a difference between maximum and minimum values of a vertical distance comprises determining whether the difference between the maximum and minimum values of the vertical distance in each of sections (a) to (c) is greater than or equal to a reference value.

3. The computer program stored on a computer-readable storage medium of claim 2, wherein the reference value is 30 mm (millimeters).

4. The computer program stored on a computer-readable storage medium of claim 1, wherein the target distance pattern graph within the gait cycle comprises two positive (+) peaks and one negative (−) peak,
the target distance pattern graph comprises (a) a section between a first peak, which is a negative peak closest to a point in time before the gait cycle, and a second peak, which is a first positive peak within the gait cycle, (b) a section between the second peak and a third peak, which is a negative peak within the gait cycle, and (c) a section between the third peak and a fourth peak, which is a last positive peak within the gait cycle, and
the determine heel-strike and toe-off points comprises:
determining a point at which a forward slope of the target distance pattern graph in section (c) is 95% or a point at which a slope of the forward slope in a section between 70% and 95% is 20% of a maximum forward slope as a heel strike; and
determining a point at which a reverse slope of the target distance pattern graph in section (c) is 85% or a point at which a slope of the reverse slope in a section between 70% and 85% is 20% of a maximum reverse slope as a toe off.

5. The computer program stored on a computer-readable storage medium of claim 4, wherein the computer program further comprises instructions executable by a processor to:
determine a point at which a forward slope of the target distance pattern graph in section (a) is 95% or a point at which a slope of the forward slope in a section between 70% and 95% is 20% of a maximum forward slope as a heel strike; and
determine a section between the heel strike in section (a) and the heel strike in section (c) as a final gait cycle.

6. The computer program stored on a computer-readable storage medium of claim 1, wherein the computer program further comprises instructions executable by a processor to:
extract a second target angle pattern, which is an angle pattern of a right or left hip joint key point, wherein the right or left hip joint key point is a hip joint key point on an opposite side of a hip joint that is a target of the first target angle pattern;
determine a second reference angle pattern for performing pattern matching with the second target angle pattern from among the plurality of angle patterns; and
generate third filtering data by performing pattern matching on the second target angle pattern and the second reference angle pattern,
wherein the determine a gait cycle comprises determining a gait cycle that satisfies all of the first to third filtering data.
